(19) United States
(12) Patent Application Publication
(10) Pub. No.: US 2003/0068393 A1
(43) Pub. Date: Apr. 10, 2003

(54) Method and composition for treating malaria

(76) Inventors: Frederick Siegfried Arkhurst, New York, NY (US); Joyce Cooper Arkhurst, New York, NY (US)

Correspondence Address: Frederick S. Arkhurst, 2235 Fifth Avenue Apt. 10-C, New York, NY, 10037, US

(21) Appl. No.: 09/973,540

(22) Filed: Oct. 9, 2001

(51) Int. Cl. A61K 35/78

(52) U.S. Cl. 424/773

(57) ABSTRACT

The invention is a composition formed from an extract of the plant Nauclea Latifolia, a method of using the composition for treating malaria and a process for preparing the composition

### FIELD OF INVENTION

This invention relates to a composition for treating malaria; a process for its preparation; and a method for its use. More specifically, this invention relates to a composition formed from an extract of the plant, Nauclea Latifolia, a method of using the composition for the treatment of malaria; and a process for preparing the composition.

### BACKGROUND

Malaria is a disease that causes the loss of millions of lives worldwide, and contributes to serious losses in man-hours of production in the economies of many countries. The problems caused by malaria have become particularly acute because of the noted increased resistance of malaria causing protozoan parasites to many of the drugs prescribed to combat the disease. For example, the most debilitating and fatal form of malaria is caused by the protozoan parasite Plasmodium Falciparum. Its resistance to drugs available for the treatment of malaria was first recognized in the United States during the Vietnam conflict, and has since been shown to be increasing in geographical range, severity and prevalence. Laboratory and epidemiological evidence now suggests that Plasmodium Falciparum has acquired multiple drug resistance that is largely independent of the chemical class of drugs to which the parasites are exposed. With multiple drug resistance in falciparum malaria, coupled with the increase in worldwide tourism, the prevention of malaria is rapidly emerging as a major health issue in the United States. Consequently, it is of critical importance to develop new drugs for the treatment of falciparum malaria.

As noted by Edward S. Ayensu in Medicinal Plants of West Africa, Reference Publications, Inc. (1978), Nauclea Latifolia is a plant known to possess anti-malarial biological activity in humans. However, no convenient and cost effective forms of medication based on this plant are currently available; therefore the need for such a medicine exists.

### DETAILED DESCRIPTION OF THE INVENTION

The present invention provides a composition made from an extract of Nauclea Latifolia for treating malaria. The invention also provides a method of using the composition for treating malaria, and a process for preparing the composition.

The present invention is an infusion made from the roots of the plant, Nauclea Latifolia, which have been cleaned and rendered free of dirt and debris; the roots are then cut up into suitably sized pieces. Although the precise size of the pieces is not critical to the preparation of the composition, pieces of about 5 centimeters (cm.) in length and about 2 cm.² in area have been found to be suitable. These pieces are fed into a bottle which is then filled with a potable alcohol, such as vodka, gin or schnapps. To obtain a concentrated form of the infusion the amount of alcohol used should just cover the level of the roots in a 750-milliliter bottle.

The roots should then be allowed to soak for a period of 15 days at room temperature—15° C. to 35° C. It has been found that by soaking the roots as prescribed the active ingredients in the roots seep into the alcohol to form a potent anti-malarial composition. Subsequently, the infusion is decanted into another bottle through a suitable filter. The roots are good for four more infusions before being discarded.

To use as prophylactic for the prevention malaria in humans, a dosage of one tablespoonful (15 milliliters) of the infusion should be ingested three times a week. For use as a cure for malaria, it has been found that a regimen of one tablespoonful of the infusion taken twice daily for three consecutive days—in a test group of human patients—has been effective. Except for a mild diuretic effect in some cases, there has been no experience of side effects in the prescribed regimen.

In contrast the majority of patients in a control group, who were given a placebo of the same color and taste as the composition, continued to suffer from malaria until given the infusion as prescribed, when they also recovered.

While particular embodiments of the present invention have been illustrated and described herein, the present invention is not limited to such illustrations and descriptions. It is apparent that changes and modifications may be incorporated and embodied as part of the present invention within the scope of the following claims.

We claim:

1. A method of making an anti-malarial composition by soaking the roots of the Nauclea Latifolia plant in potable alcohol to obtain an infusion comprising a therapeutically effective plant extract; and separating the roots from said composition.

2. The method according to claim 1, wherein soaking includes maintaining the roots and the potable alcohol at a temperature from 15° C. to 70° C.

3. The method according to claim 1, wherein soaking includes maintaining the roots and the potable alcohol at a temperature from 15° C. to 50° C.

4. The method of claim 1 further comprises providing the roots of the Nauclea Latifolia plant in pieces of less than 10 cm. in length and less than 4 cm.² in cross-sectional area.

5. A composition for treating malaria comprising a therapeutically effective amount of extract of Nauclea Latifolia roots; and a potable alcohol.
